(19) United States
(12) Patent Application Publication
(10) Pub. No.: US 2021/0378858 A1
(43) Pub. Date: Dec. 9, 2021

(54) CHIN POSITIONING APPARATUS

(71) Applicant: William F. Urmey, Larchmont, NY (US)

(72) Inventor: William F. Urmey, Larchmont, NY (US)

(21) Appl. No.: 17/340,160

(22) Filed: Jun. 7, 2021

Related U.S. Application Data
(60) Provisional application No. 63/036,036, filed on Jun. 8, 2020.

(51) Int. Cl. A61F 5/56; A41D 13/12

(52) U.S. Cl. CPC A61F 5/56; A41D 1/02; A41D 13/1245

(57) ABSTRACT

An adjustably positioned foam chin support maintains the chin in a position precluding or minimizing upper airway obstruction. The chin support is seated within a garment with a portion of the support extending above the garment neck for engaging the user's chin. A garment neckband includes tangentially extending tension cords. Each cord extends through an opening in a front panel of the garment. The ends of the cords are pulled to tension the neckband against the back of the user's neck and apply upward compressive force to position the chin support. A cord lock fixes the tension of the cords. A pair of pouches is affixed to the interior of the garment at the mid-clavicular lines, spaced below the cord openings. The lower corners of the chin support are seated in the pouches.

### RELATED APPLICATIONS

This application claims the benefit of Provisional Application No. 63/036036 filed 8 Jun. 2020, the entirety of which is incorporated herein by reference.

### BACKGROUND OF THE INVENTION

### 1. Field of the Invention

This invention relates generally to management of the upper airway to minimize obstructions and related conditions such as, snoring and apnea, and more particularly to a chin positioning apparatus

### 2. Antecedents of the Invention

Snoring and upper airway obstruction during sleep are extremely common in adults. Upper airway obstruction has been treated with positional therapy, chin straps, electrical stimulation systems, pillows that elevate the head, continuous positive airway systems with a fitted mask, jaw elevation devices, such as those disclosed in U.S. Pat. No. 4,700,697 and US Publication Nos. 2019/0015245, 2012/0234330, 2012/0216820 and 2010/0294284 and dental appliances, such as those disclosed in U.S. Pat. Nos. 10,327,944 and 10,799,389, Many of these proposed devices were not commercially successful because they were uncomfortable and did not accommodate multiple positions (supine, lateral decubitus, sitting, semi-recumbent, and prone), required sterilization, were not compatible with continuous positive airway pressure devices and were relatively costly.

Intra-operative sedation or sedation for office based procedures, e.g., colonoscopy or biopsy, often result in upper airway obstruction in those patients with anatomy that predisposes them to snoring, airway obstruction, or apnea (cessation of breathing). More serious upper airway obstruction during sleep results in obstructive sleep apnea (OSA) which is characterized by periods of apnea and deoxygenation. OSA is a highly prevalent condition throughout the world. The incidence of OSA has been estimated at greater than 50% in many countries with estimates of over one billion adults affected with the condition worldwide.

Continuous positive airway pressure (CPAP) is the gold standard for treatment of patients with OSA. However, many patients do not tolerate and therefore cannot use CPAP devices. In addition, CPAP systems may become contaminated with bacteria, viruses, or other micro-organisms, resulting in patient infections and pneumonia.

During the Covid19 viral pandemic, scientific evidence revealed that CPAP and other positive pressure assisted breathing leads to aerosolization of viral particles and potential spread of infection. Such techniques of assisted ventilation have been discouraged in patients with viral pneumonia and the recommendation has been made to proceed directly to endotracheal intubation and placement of the patient onto a mechanical ventilator. Further, there is evidence that ventilation leads to better distribution of ventilation and perfusion in the lungs, leading to better outcomes. It has also been suggested that patients may do worse if intubation and positive pressure ventilation is started before absolutely necessary in such patients.

### SUMMARY OF THE INVENTION

An adjustably positioned compressible foam chin support is seated within a vest or jacket which may comprise a disposable single use garment formed of paper or nonwoven fabric. Alternately, the garment may be reusable and formed of washable fabric.

The garment includes a neckband having a pair of tangentially extending cords which are separately attached. Each cord extends through a garment opening registered with the mid-clavicular lines. Both ends of the cord are pulled to tension and adjust h foam chin support. A cord lock fixes the tension of the cord. Optionally, a single cord is threaded through the neckband.

A pair of pouches is affixed to the interior of the garment at the mid-clavicular spaced below the cord openings. The lower corners of the chin support are seated in the pouches.

From the foregoing compendium, it will be appreciated that an aspect of the present invention is to provide an improved chin positioning apparatus for alleviation of upper airway obstruction and related conditions of the general character described which is not subject to the aforementioned disadvantages of the antecedents of the invention.

A feature of the present invention is to provide an improved chin positioning apparatus for alleviation of upper airway obstruction and related conditions of the general character described which decreases or minimizes upper airway obstruction, snoring, or symptoms of sleep apnea by allowing the maintenance of natural negative pressure inspiration without the apparatus for alleviation of upper airway obstruction and related conditions of the general character described which may be employed need for continuous positive airway pressure (CPAP).

A consideration of the present invention is to provide an improved chin positioning during regional anesthesia or office-based surgical procedures requiring sedation of a patient.

An additional aspect of the present invention is to provide an improved chin positioning apparatus for alleviation of upper airway obstruction and related conditions of the general character described which allows for the measurement and optimal adjustment of the tension of a chin support and therefore allows for a reproducible support for a given subject who uses it more than once.

Another feature of the present invention is to provide an improved chin positioning apparatus for alleviation of upper airway obstruction and related conditions of the general character described which is inexpensive.

A still further consideration of the present invention is to provide an improved chin positioning apparatus for alleviation of upper airway obstruction and related conditions of the general character described which is disposable so as not to require cleaning or sterilization between uses at home or in the medical setting, and, therefore, cross contamination between patients is avoided.

To provide an improved chin positioning apparatus for alleviation of upper airway obstruction and related conditions of the general character described which is suitable for use in conjunction with CPAP to minimize positive airway pressure and minimize potential cardiac and hemodynamic side effects is a further aspect of the present invention.

An additional feature of the present invention is to provide an improved chin positioning apparatus for alleviation of upper airway obstruction and related conditions of the general character described wherein chin elevation may be easily adjusted and controlled by a user or health care provider.

### BRIEF DESCRIPTION OF THE DRAWINGS

In the accompanying wherein one of the possible exemplary embodiments of the invention is shown:

FIG. 1 is a front elevational view of a garment for holding a foam chin support in accordance with the invention and showing a posterior neckband from which a pair of cords extend;

FIG. 2 is a front elevational view of the garment with a zipper opened to reveal one of the cords extending through an opening in a right front panel of the garment and an interior pouch in which a corner of the foam chin support seats;

FIG. 3 is an enlarged cross sectional view through the garment taken substantially along the plane 3-3 of FIG. 2, with the user's chin correctively positioned with the foam chin support; also illustrating the cords applying appropriate tension for proper chin engagement and the internal pouches for maintaining the position of the chin support relative to the garment;

FIG. 4 is a reduced scale perspective view of the foam chin support and showing a concave channel in its upper surface for engaging the user's chin and larger convex cut away portion at the bottom to avoid pressure on anatomical structures of the neck;

FIG. 5 is an enlarged scale sectional view through the left front panel, the same being taken substantially along the plane 5-5 of FIG. 2, and showing an internal pouch formed of two strata joined along a vertical seam and a web spanning between the strata.

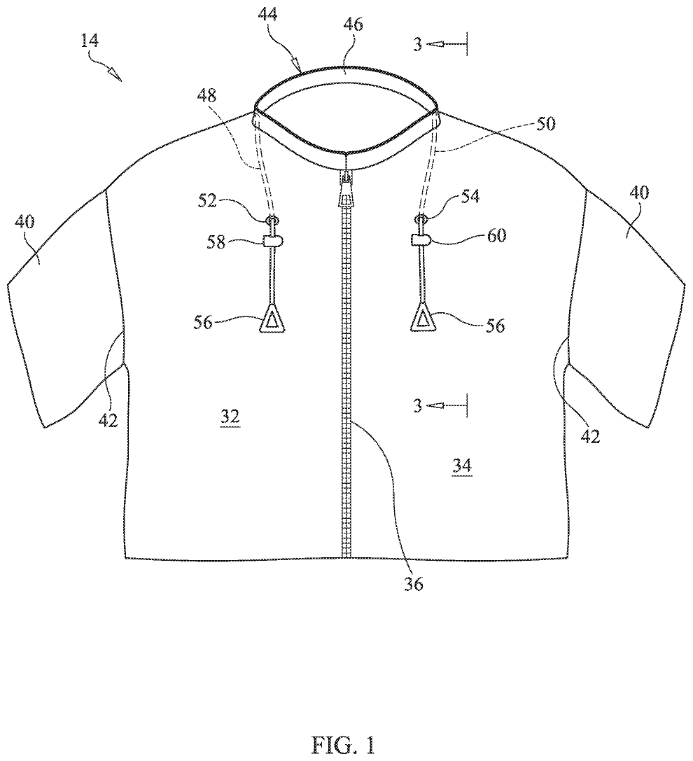

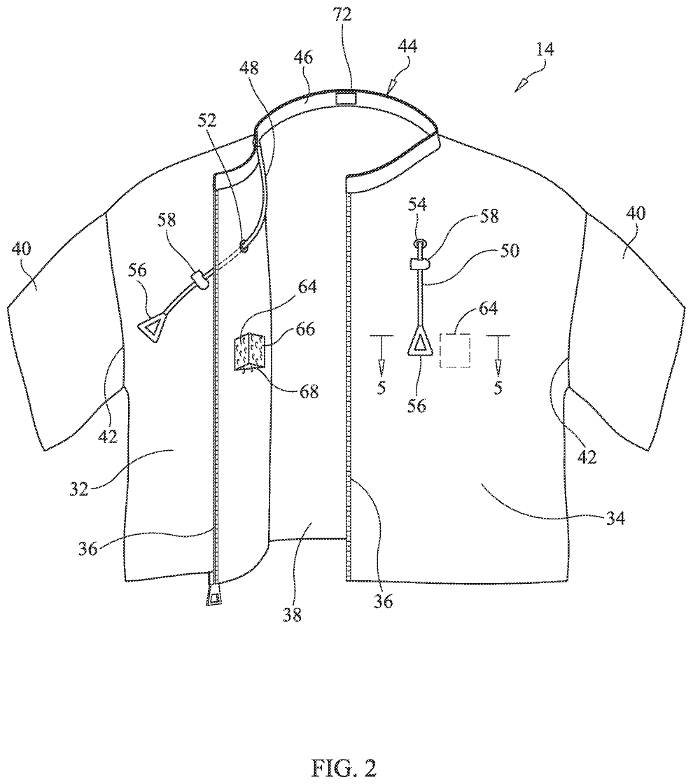

### DESCRIPTION OF THE PREFERRED EMBODIMENT

The present invention will now be described in detail with reference to the drawings, which are provided as illustrative examples of the invention so as to enable those skilled in the art to practice the invention. Notably, the figures and examples below are not meant to limit the scope of the present invention to a single embodiment, but other embodiments are possible by way of interchange of some or all of the described or illustrated elements.

Moreover, where certain elements of the present invention can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein.

Applicant does not intend for any term in the specification or claims to be ascribed an uncommon or special meaning unless explicitly set forth as such. Further, the present invention encompasses present and future known equivalents to the known components referred to herein by way of illustration,

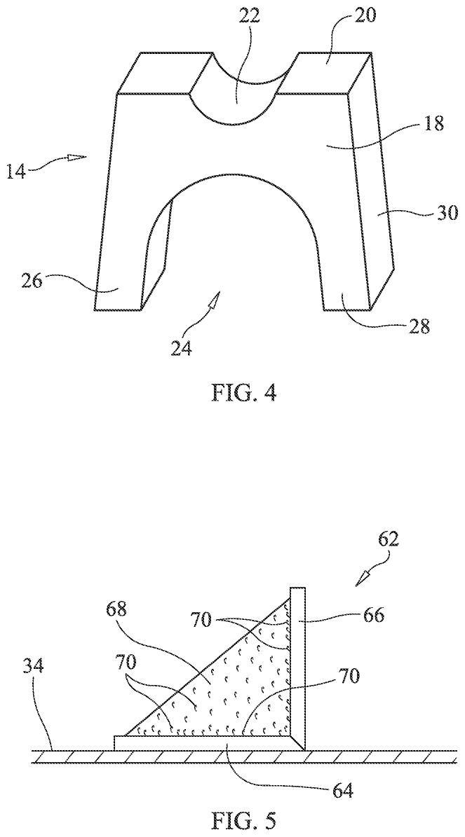

Referring now in detail to the drawings wherein like numerals denoted like components throughout, the reference numeral 10 denotes a chin positioning apparatus for alleviation of upper airway obstruction and related conditions constructed in accordance with and embodying the present invention. The apparatus includes a compressible foam chin support 12, illustrated in FIGS. 4 and 5 and an upper body garment 14, such as a jacket, vest or waistcoat for positioning the foam chin support to engage a user's chin.

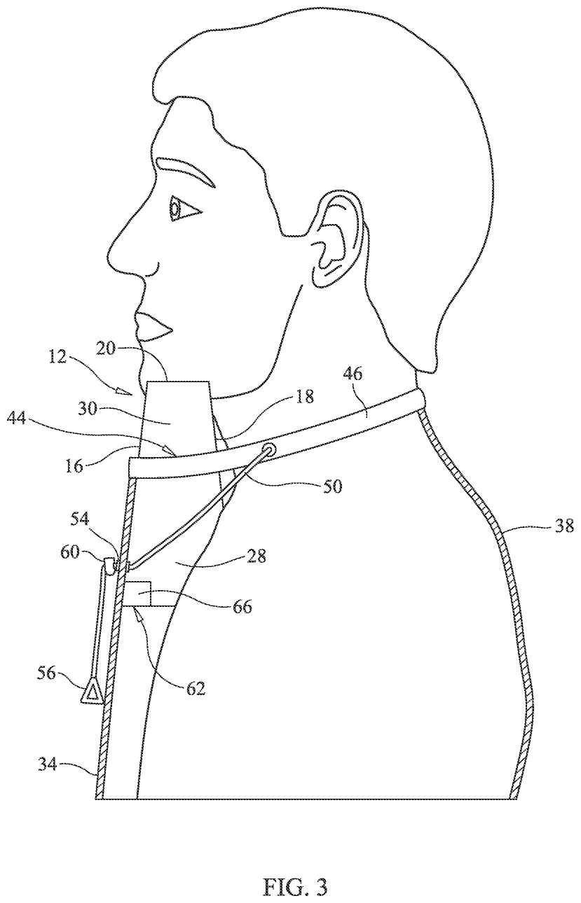

Referring to FIGS. 3 and 4, the foam chin support 12 may comprise open cell polyurethane foam shaped substantially as a solid rectangle. The chin support includes planar front and rear faces 16, 18, a planar top 20 having a central concave chin rest 22 and an inverted U shaped cavity 24 at the bottom, which forms a pair of legs 26, 28. The cavity 24 assures that when the foam chin support us positioned against the chest and the neck, there will be no interference with the shaped portion that is removed (FIG. 4), so as not to apply pressure against the important anatomical structures of the neck, e.g., carotid arteries, jugular veins, carotid bodies, etc. A pair of opposed side walls 30 may extend parallel to one another or may taper in a superior direction.

The garment 14 includes a front formed of right and left front panels 32, 34 which are separable along a central zipper 36. Other garment fasteners, e.g., hook and loop fastener strips, snaps, buttons, ties, etc., can be employed. A rear panel 38 joins the front panels 32, 34. If configured as a jacket, the garment 14 includes sleeves 40 which extend from armholes 42. If configured without sleeves, only armholes 42 are provided.

A neck opening 44 includes a polyurethane foam padded neckband 46 configured to engage the back of the user's neck. A pair of tension cords 48, 50 are fixed to opposite ends of the neckband. The tension cords 48, 50 extend downwardly along the inner faces of the respective front panels 32, 34 and through grommet reinforced exit holes 52, 54 in the front panels 32, 34. The exit holes 52, 54 are located high in the right and left mid-clavicular lines.

A pull knob 56 is secured at the end of each tension cord. Affixed to each tension cord between the pull knob and the holes 52, 54 there is a cord lock 58, 60, such as that described in U.S. Pat. No. 7,254,871 or US Patent Publication 20160037846, both of which are incorporated herein in their entireties by reference. The tension within the tension cords 48, 50 is adjusted by pulling the knobs 56 and when the desired tension is attained, the cord locks 58, 60 are slid upwardly along their tension cords to abut their respective holes 52, 54 and then locked in position. Optionally, the neckband 46 may comprise a hollow padded sleeve through which a single cord is threaded, with two end portions of the cord extending through the grommet reinforced exit holes. The applied tension can be measured and duplicated to reproduce the settings for repeated usage. A strain gauge 72 may be provided at the rear center of the neckband to measure the force applied to tighten the tension cords, for example, or to measure the force applied against the rear of the user's neck.

As illustrated in FIG. 3, the foam chin support 12 is inserted between the front panels and the user's chin, neck and chest. The user's chin seats in the chin rest 22; the portion of the front face 16 positioned beneath the neck opening 44 is engaged by the panels 32, 34. With tension applied through locked tension cords, the rear face 18 is compressed against the user's chest.

On the interior of each front panel 32, 34, there is provided a foldable pouch 62 for engaging and retaining the foam chin support 12 relative to the garment 14. The pouch 62 comprises a first rectangular or square stratum 64 adhered to each of the front panels 32, 34 by peripheral stitching or adhesive and a second stratum 66 joined to the first stratum 64 along a common vertical edge. The second stratum 66 may be folded against the first stratum 62 when the garment 14 is not in use. There is additionally provided a flexible web 68, which spans between the strata 62, 64 and which folds when the strata are folded together.

As will be noted from an examination of FIG. 3, when the foam chin support 12 is inserted between the front panels and the user's chin, neck and chest, the face 16 of the leg 28 engages the first stratum 64 while the side wall 30 of the chin support 12 engages the second stratum. Additionally, the web 68 engages the bottom of the leg 26.

Pursuant to the invention, the interior faces of the strata 62, 64 and the web 66 are coated or covered with material having a plurality of hook elements 70, such as that employed with hook and loop fasteners. The hook elements adhere to the open cell foam structure of the foam chin support 12 for enhanced securement.

Positioning and support of the chin is effected by a leveraging system. Static cephalad forces are exerted through the garment 14 which is fixed in the axillary folds, through engagement by the armholes 42 bilaterally, as well as the bilateral pouches 62. The opposite caudal static tension force, which is adjusted by pulling on the tension cords 48, 50, determines the static pressure in the opposite direction on the chin. An adjustable fulcrum is provided by the neckband at the level of the “Cervical Prominens”, i.e., C-7.

Importantly, this static balance of forces and anatomical relationship is not substantially changed by alteration in the patient's position to lateral decubitus or prone positions. The chin positioning apparatus 10 can be easily applied by the patient at home, without the assistance of a healthcare professional.

The particular combinations of elements and features in the above-detailed embodiment are exemplary only; the interchanging and substitution of these teachings with other teachings in this application are also expressly contemplated. As those skilled in the art will recognize, variations, modifications, and other implementations of what is described herein can occur to those of ordinary skill in the art without departing from the spirit and the scope of the invention as claimed.

Further, in describing the invention and in illustrating an embodiment of the invention in the figures, specific terminology, numbers, dimensions, materials, etc., are used for the sake of clarity. However the invention is not limited to the specific terms, numbers, dimensions, materials, etc. so selected, and each specific term, number, dimension, material, etc., at least includes all technical and functional equivalents that operate in a similar manner to accomplish a similar purpose. Use of a given word, phrase, number, dimension, material, language terminology, product brand, etc. is intended to include all grammatical, literal, scientific, technical, and functional equivalents. The terminology used herein is for the purpose of description and not limitation.

Having described the preferred embodiment of the invention, it will now become apparent to one of ordinary skill in the art that other embodiments incorporating the concept may be used. Moreover, those of ordinary skill in the art will appreciate that the embodiment of the invention described herein can be modified to accommodate and/or comply with changes and improvements in the applicable technology and standards referred to herein.

Variations, modifications, and other implementations of what is described herein can occur to those of ordinary skill in the art without departing from the spirit and the scope of the invention as claimed. It is felt therefore that these embodiments should not be limited to the disclosed embodiment but rather should be limited only by the spirit and scope of the appended claims.

Having thus described the invention, there is claimed as new and desired to be secured by Letters Patent:

1. A chin positioning apparatus for minimizing upper airway obstructions, the apparatus comprising a foam support configured for engaging the lower portion of a user's chin and an upper body garment, the upper body garment including a front, a rear, armholes and a neck opening, a portion of the foam support extending above the neck opening, a portion of the foam support extending beneath the neck opening and being covered by the front, a rear portion of the neck opening comprising a neckband for engagement against a rear portion of a user's neck, a pair of tension members extending from the neckband, each of the tension members extending through an opening in the front, whereby when tension is applied to the tension members, the upper body garment exerts cephalad forces against the foam support to appropriately position the user's chin for maximizing airway efficiency.

2. The chin positioning apparatus for minimizing upper airway obstructions in accordance with claim 1 wherein the upper body garment is selected from the group consisting of a vest, a jacket and a waistcoat.

3. The chin positioning apparatus for minimizing upper airway obstructions in accordance with claim 1 wherein the front of the upper body garment includes a pair of panels, each panel including one of the openings, the panels being selectively joined by a fastener selected from the group consisting of a zipper, hook and loop fastener strips, snaps, buttons and ties.

4. The chin positioning apparatus for minimizing upper airway obstructions in accordance with claim 1 wherein the foam support comprises open cell polyurethane foam.

5. The chin positioning apparatus for minimizing upper airway obstructions in accordance with claim 1 further including a pair of pouches fixed to the inside of the front of the upper body garment, the pouches being configured to capture and retain portions of the foam support.

6. The chin positioning apparatus for minimizing upper airway obstructions in accordance with claim 3, further including a pouch fixed to the inside of each panel, the pouches being configured to capture and retain portions of the foam support.

7. The chin positioning apparatus for minimizing upper airway obstructions in accordance with claim 1 wherein the foam support includes an upper surface having a concave chin rest and a lower portion having an inverted “U” shaped transverse cut away for avoidance of anatomical structures of the user's neck when compressive forces are exerted against the foam support.

8. The chin positioning apparatus for minimizing upper airway obstructions in accordance with claim 1 wherein the pair of tension members comprise end portions of a single cord, the cord being threaded through the neckband.

9. The chin positioning apparatus for minimizing upper airway obstructions in accordance with claim 6 wherein each pouch comprises a pair of strata having lower edges, one stratum being fixed to the inside of one panel, the other stratum being joined to the one stratum along a common fold line, the pouch further comprising a web joining the lower edges of the strata.

10. The chin positioning apparatus for minimizing upper airway obstructions in accordance with claim 1 further including a strain gauge operatively connected to the tension members

11. A chin positioning apparatus for minimizing upper airway obstructions, the apparatus comprising a foam support configured for engaging the lower portion of a user's chin and an upper body garment, the foam support including a front face, a rear face, a top having a concave chin rest, a bottom and a cavity extending between the front face and rear face from the bottom toward the top, the cavity defining a pair of legs at the bottom, the apparatus further comprising an upper body garment, the upper body garment including a front, a rear, and a neck opening, a portion of the foam support extending above the neck opening, a portion of the foam support extending beneath the neck opening, the upper body garment further including tensioning members for exerting compressive and cephalad forces against the foam support to position the user's chin.

12. The chin positioning apparatus for minimizing upper airway obstructions in accordance with claim 11 the wherein the cavity is shaped as an inverted letter “U”.

13. The chin positioning apparatus for minimizing upper airway obstructions in accordance with claim 11 further including a pair of pouches fixed to the inside of the front of the upper body garment, each pouch being configured to capture and engage a leg of the foam support.

14. The chin positioning apparatus for minimizing upper airway obstructions in accordance with claim 13 wherein the pouches are lined with hook members for engaging and retaining the foam support.

15. The chin positioning apparatus for minimizing upper airway obstructions in accordance with claim 11 the wherein the tensioning members comprise a pair of cords which extend through openings in the front, the chin positioning apparatus further including cord lock adjustably fixed to each cord, the cord locks being in abutment against the openings in the front.

16. The chin positioning apparatus for minimizing upper airway obstructions in accordance with claim 13, the upper body garment further including armholes, the upper body garment exerting bilateral cephalad forces against the chin support through engagement by the armholes and the pouches.

17. A chin positioning apparatus for minimizing upper airway obstructions, the apparatus comprising a foam support configured for engaging the lower portion of a user's chin, a front face, a rear face, a top having a concave chin rest and a bottom, the apparatus further comprising an upper body garment, the upper body garment including a front, a portion of the foam support extending above the front, a portion of the foam support extending behind the front, the apparatus further including a tensioning member configured extend around the rear of the user's neck, the tensioning member having end portions extending through openings in the front, the tensioning member exerting compressive and cephalad forces through the front and against the foam support when tensioned.

18. The chin positioning apparatus for minimizing upper airway obstructions in accordance with claim 17 further including a gauge for measuring the tension applied to the tensioning member.

19. The chin positioning apparatus for minimizing upper airway obstructions in accordance with claim 17 wherein the front is comprised of a pair of panels, each panel including an opening, the panels being selectively joined by a fastener selected from the group consisting of a zipper, hook and loop fastener strips, snaps, buttons and ties.

20. The chin positioning apparatus for minimizing upper airway obstructions in accordance with claim 16 further including a pair of pockets fixed to the inside of the front, the pouches being configured to engage and retain portions of the foam support which extend behind the front.
